(19) United States
(12) Patent Application Publication
(10) Pub. No.: US 2012/0059024 A1
(43) Pub. Date: Mar. 8, 2012

(54) DRUG ABUSE DETERRENT, METHODS AND COMPOSITIONS

(75) Inventors: Ian Robert Chambers Cooke, Hawthorn (AU); Colin Stanley Goodchild, Malvern (AU); Claudia C. Gregorio-King, Abbotsford (AU)

(73) Assignee: RELEVARE AUST. PTY LTD, Abbotsford (AU)

(21) Appl. No.: 13/147,185

(22) PCT Filed: Jan. 28, 2010
(86) PCT No.: PCT/AU2010/000084

§ 371 Date: Nov. 10, 2011

Related U.S. Application Data
(60) Provisional application No. 61/148,845, filed on Jan. 30, 2009.

(51) Int. Cl. A61K 31/485; A61K 31/451; A61P 25/04; A61K 31/27; A61K 31/4535; A61K 31/4468; A61K 31/44

(52) U.S. Cl. 514/282; 514/329; 514/289; 514/330; 514/352; 514/479; 514/326

(57) ABSTRACT

The present invention relates generally to the field of pain management. More particularly, the present invention relates to analgesic combinations of an opioid and flupirtine or retigabine which reduce the risk of substance abuse in pain management. Such combinations are referred to as abuse-deterrent fixed dose combinations (FDC).

### FIELD

The present invention relates generally to the field of pain management. More particularly, the present invention relates to analgesic combinations of an opioid and flupirtine or retigabine which reduce the risk of substance abuse in pain management. Such combinations are referred to as abuse-deterrent fixed dose combinations (FDC).

### BACKGROUND

Bibliographic details of the publications referred to by author in this specification are collected alphabetically at the end of the description.

Reference to any prior art in this specification is not, and should not be taken as, an acknowledgment or any form of suggestion that this prior art forms part of the common general knowledge in any country.

Opioid analgesics, also known as narcotic analgesics, are pain relievers that act on the central nervous system. Like all narcotics, opioids are a target of abuse due to the euphoria inducing side-effect of the compounds. Opioids induce feelings of euphoria by affecting the brain regions that mediate the perception of pleasure. This feeling is often intensified for those who abuse opioids when taken in amounts greater or via route other than that recommended. For example, nasal inhalation or injection of OxyContin, enhances its euphoric effect, which therefore increases the risk of adverse medical and/or social consequences, such as opioid over use.

Due to their powerful pain relieving abilities, opioids are widely manufactured and readily available. It is this availability, in combination with their mind altering effects, which have made opioids a target for substance abuse.

Accordingly, there is a need to develop dosage forms of opioids which not only provide an analgesic effect, but which reduce the risk of substance abuse.

### SUMMARY

The present invention is directed to methods and compositions for treating, alleviating, preventing, diminishing or otherwise ameliorating the symptoms associated with pain in a subject while reducing incentive of recipients to abuse the recommended doses in order to enhance or prolong the euphoric effects. More particularly, the present invention contemplates abuse-deterrent fixed dose combinations (FDC) of an opioid and one of flupirtine or retigabine, and methods of using same to manage pain without enticing a subject to engage in substance abuse.

The present invention provides, therefore, an abuse-deterrent FDC comprising an analgesically effective amount of an opioid and flupirtine or retigabine. Flupirtine and retigabine, because of their sedating and dysphoric activity and other non-life threatening side-effects such as nausea and vomiting, when used at high doses, inhibit or discourage abuse of the FDC, including reducing the enticement to use the FDC to induce a state of euphoria rather than its intended use as an analgesic. The sedative and dysphoric nature of the high dose flupirtine or retigabine counteracts the opioid induced euphoria, while the adverse effects such as vomiting and nausea exert a negative effect on any over consumption/reward pathways that would otherwise reinforce the abuse behaviour.

Accordingly, one aspect of the present invention contemplates a method for inducing an analgesic response to pain in a subject, the method comprising administering to the subject of an abuse-deterrent FDC comprising an amount of an opioid and flupirtine or retigabine which is effective to reduce the level of, or otherwise ameliorate the sensation of pain.

More particularly, the present invention provides a method for inducing an analgesic response to pain in a subject whilst reducing the enticement for substance abuse, the method comprising administering to the subject an abuse-deterrent FDC comprising an amount of an opioid and flupirtine or retigabine which is effective to reduce the level of, or otherwise ameliorate the sensation of, pain.

The present invention further is directed to a method for inducing an analgesic response to pain in a subject said method comprising administering to the subject an amount of an opioid and an agent selected from flupirtine and retigabine which is effective to reduce the sensation of pain whilst reducing the enticement for substance abuse.

Even more particularly, the present invention is directed to a method of inducing an analgesic response to pain in a subject, the method comprising the administration of an abuse-deterrent FDC providing between 0.1 mg and 200 mg of morphine or an equivalent dose of another opioid, as determined using a table of equivalent dose factors (see Table 1), and between 25 mg and 1000 mg of flupirtine or 10 mg and 500 mg of retigabine.

The FDC also comprises flupirtine or retigabine in an amount that provides an adverse experience in the subject who aims to abuse the FDC to achieve an opioid induced euphoria, by taking either more than the recommended dose or by taking it by a route other than intended by the manufacturer.

The flupirtine or retigabine is present in an amount that provides an adverse experience and/or euphoria inhibition in a subject when the subject takes at least 2 times the recommended dose of the FDC.

An “adverse experience” includes range of experiences from mildly negative to a significant negative experience. Generally, the experience is sufficiently unpleasant and/or euphoria inhibiting so as to deter the future abuse of the FDC

In a further aspect, the present invention provides an abuse-deterrent composition comprising an analgesic amount of an opioid and flupirtine or retigabine. The composition may further comprise one or more pharmaceutically acceptable carriers, diluents and/or excipients.

Yet another aspect relates to the use of an opioid and flupirtine or retigabine in the manufacture of an abuse-deterrent FDC for inducing an analgesic response in the treatment of pain.

A further aspect of the invention provides the use of abuse-deterrent FDC in the manufacture of a medicament for inducing an analgesic response in the treatment of pain. In a further aspect, the FDC comprises an analgesically effective amounts of two or more opioids in combination with flupirtine or retigabine in an amount that provides an adverse experience in a subject.

Pain management protocols which reduce the incentive to abuse opioid intake, including point of care therapeutic protocols for controlling pain or the sensation of pain, are also provided herein. The protocols include assessing a subject for pain type or causation of pain and providing to the subject the abuse-deterrent FDC comprising an opioid and flupirtine or retigabine.

Hence, the present invention provides a pain management protocol with a reduced enticement for substance abuse, said protocol comprising administering to a subject in need of pain management an amount of an opioid and an agent selected from flupirtine and retigabine which is effective to reduce the sensation of pain whilst reducing the enticement for substance abuse.

### DETAILED DESCRIPTION

Throughout the specification and the claims which follow, unless the context requires otherwise, the word “comprise”, and variations such as “comprises” and “comprising” will be understood to imply the inclusion of a stated integer or step or group of integers or steps but not the exclusion of any other integer or step or group of integers or steps.

Reference to the prior art in this specification is not, and should not be taken as an acknowledgement or any form of suggestion that this prior art forms part of the common general knowledge in any country.

The singular forms “a”, “an” and “the” include the plural aspects unless the context clearly dictates otherwise. Thus, for example, reference to “a pain” includes single or multiple types of pain; reference to “an opioid” includes a single opioid, as well as two or more opioids; reference to “the invention” includes one aspect or multiple aspects of the invention.

Terms such as “opioid euphoric-inhibiting amount”, “an analgesically effective amount”, “effective amount” and “therapeutically effective amount” as used herein mean a sufficient amount of an opioid combined with flupirtine or retigabine to provide the desired therapeutic or physiological effect or outcome, which includes the achievement of pain reduction without an enticement to abuse the combination to induce a euphoric effect. The exact amount required will vary from subject to subject, depending on the age, gender and general condition of the subject, mode of administration and the like.

As used herein, an “effective amount” refers to an amount of active agent that provides the desired analgesic activity when administered according to a suitable dosing regime. The amount of active agent is generally an amount that provides the desired analgesic activity. In one aspect, this occurs without causing overt sedation or dose limiting side-effects or drug tolerance. Dosing may occur at intervals of several minutes, hours, days, weeks or months. Suitable dosage amounts and regimes can be determined by the attending physician or veterinarian.

The terms “treating” and “treatment” as used herein refer to the act of administering to a subject or exposing the subject to a pain management protocol comprising an analgesic composition which is unlikely to encourage substance abuse. It includes a reduction in severity and/or frequency of pain, elimination of symptoms and/or underlying cause of pain, prevention of the occurrence of pain associated with a condition or its underlying cause and improvement or remediation or amelioration of pain. Increasing the recommended treatment dose leads to an unpleasant and/or adverse experience and/or a euphoria inhibiting effect which reduces the enticement to abuse the analgesic composition.

A “subject” as used herein refers to a human who can benefit from the analgesic compositions and methods, including pain management protocols, of the present invention.

Accordingly, the present invention provides a method for inducing an analgesic response to pain in a subject, the method comprising administering to the subject an abuse-deterrent FDC comprising an amount of an opioid and flupirtine or retigabine which is effective to reduce the level of, or otherwise ameliorating the sensation of, pain.

In a related aspect, the present invention contemplates a method for inducing an analgesic response to pain in a subject whilst reducing the enticement for substance abuse, the method comprising administering to the subject an abuse-deterrent FDC comprising an amount of an opioid and flupirtine or retigabine which is effective to reduce the level of, or otherwise ameliorate the sensation of pain.

The present invention further provides a method for inducing an analgesic response to pain in a subject said method comprising administering to the subject an amount of an opioid and an agent selected from flupirtine and retigabine which is effective to reduce the sensation of pain whilst reducing the enticement for substance abuse.

The flupirtine or retigabine is present in an amount which, when the FDC is administered in its recommended dose, causes no or minimal adverse effects. The flupirtine or retigabine is also present in an amount which, when the FDC is administered in its recommended dose, may minimise the possibility of obtaining opioid induced euphoria. However, when a subject takes more than the recommended dosage of the FDC (for example, twice the recommended dose), or takes the FDC by a route other than as intended with the possibility of obtaining opioid induced euphoria, non-life threatening adverse reactions or effects result. Adverse reactions include, without being limited to, sedation, tiredness, somnolence, sleepiness, dizziness, nausea, vomiting, abdominal pain, sweating, depression, teariness (crying fits), headache, tremor, restlessness, nervousness, confusion, disorientation, dysphoria, blurred vision, tachycardia. The adverse reaction is a range of experiences from a mildly negative to a significantly unpleasant experience so as to provide reduced enticement to use the FDC in an abusive manner, such as to induce euphoria.

Opioid analgesics suitable for use in the FDC of the present invention include, without being limited to, oxycodone, hydromorphone, morphine, hydrocodone, fentanyl, oxymorphone, codeine, alfentanil, allylprodine, alphaprodine, anileridine, benzylmorphine, bezitramide, buprenorphine, butorphanol, clonitazene, desomorphine, dextromoramide, dezocine, diampromide, diamorphone, dihydrocodeine, dihydromorphine, dimenoxadol, dimepheptanol, dimethylthiambutene, dioxaphetyl butyrate, dipipanone, eptazocine, ethoheptazine, ethylmethylthiambutene, ethylmorphine, etonitazene, heroin, hydroxypethidine, isomethadone, ketobemidone, levorphanol, levophenacylmorphan, lofentanil, meperidine, meptazinol, metazocine, methadone, metopon, myrophine, narceine, nicomorphine, norlevorphanol, normethadone, nalorphine, nalbuphene, normorphine, norpipanone, opium, oxymorphone, papvereturn, pentazocine, phenadoxone, phenomorphan, phenazocine, phenoperidine, piminodine, piritramide, propheptazine, promedol, properidine, propoxyphene, sufentanyl, tapentadol, tilidine or tramadol as well as their pharmaceutically acceptable salts. These are referred to as being “active compounds” or terms such as agents, medicaments and the like.

In certain aspects, the FDC contains two or more opioids.

The salts of the active compounds of the invention can be pharmaceutically acceptable, but it will be appreciated that non-pharmaceutically acceptable salts also fall within the scope of the present invention, since these are useful as intermediates in the preparation of pharmaceutically acceptable salts. Examples of pharmaceutically acceptable salts include salts of pharmaceutically acceptable cations such as sodium, potassium, lithium, calcium, magnesium, ammonium and alkylammonium; acid addition salts of pharmaceutically acceptable inorganic acids such as hydrochloric, orthophosphoric, sulfuric, phosphoric, nitric, carbonic, boric, sulfamic and hydrobromic acids; or salts of pharmaceutically acceptable organic acids such as acetic, propionic, butyric, tartaric, maleic, hydroxymaleic, fumaric, citric, lactic, mucic, gluconic, benzoic, succinic, oxalic, phenylacetic, methanesulphonic, trihalomethanesulfphonic, toluenesulphonic, benzenesulphonic, salicyclic, sulphanilic, aspartic, glutamic, edetic, stearic, palmitic, oleic, lauric, pantothenic, tannic, ascorbic and valeric acids.

The dosage range of the opioid analgesic ranges from between 0.1 mg and 200 mg for orally administered morphine and equivalent dosage ranges for morphine administered via other routes (as determined from a table of mean equivalent daily dose factors [MEDD factors] for morphine and other opioid drugs) and between equivalent dosage ranges for other opioid drugs other than morphine administered orally or by other routes. A suitable table of MEDD-Factors for determining equivalent doses of morphine and other opioid drugs administered by various routes is shown in Table 1. Table 1 is published on the Internet (www.palliative.org/PC/ClinicalInfo/AssessmentTools/MeanEquivalent%20for%20program%20v3.pdf) at the official website of the Regional Palliative Care Program in Edmonton Alberta (www.palliative.org). Similar tables of mean equivalent daily doses of opioid analgesics and MEDD-factors are published elsewhere in the medical literature and on the official websites of other reputable medical organisations and clinical support groups.

TABLE 1
Mean Equivalent Daily Dose (MEDD) Conversion Table

| Medication | Route | MEDD-Factor |
|---|---|---|
| Codeine | IM | 0.1 |
| Codeine | PO | 0.05 |
| Codeine | R | 0.05 |
| Codeine | SC | 0.1 |
| Diamorphine | PO | 0.65 |
| Diamorphine | SC | 1.3 |
| Diamorphone | EP | 3.9 |
| Diamorphone | IT | 39 |
| Fentanyl | EP | 0.3 |
| Fentanyl | IT | 3 |
| Fentanyl | PO | 0.05 |
| Fentanyl | SL | 0.05 |
| Fentanyl | IV | 0.1 |
| Fentanyl | SC | 0.1 |
| Fentanyl | TD | 0.1 |
| Hydrocodone | PO | 0.4 |
| Hydromorphone | IM | 5 |
| Hydromorphone | IV | 5 |
| Hydromorphone | PO | 2 |
| Hydromorphone | SC | 5 |
| Hydromorphone | EP | 15 |
| Hydromorphone | IT | 150 |
| Levo-dromoran | SC | 5 |
| Levo-dromoran | 0 | 0.25 |
| Meperidine | EP | 0.3 |
| Meperidine | IT | 3 |
| Meperidine | IM | 0.1 |
| Meperidine | IV | 0.1 |
| Meperidine | PO | 0.05 |
| Meperidine | SC | 0.1 |
| Methadone | EP | 24 |
| Methadone | IT | 240 |
| Methadone | IV | 8 |
| Methadone | PO | 4 |
| Methadone | R | 4 |
| Methadone | SC | 8 |
| Morphine | EP | 3 |
| Morphine | IT | 30 |
| Morphine | IM | 1 |
| Morphine | IV | 1 |
| Morphine | PO | 0.4 |
| Morphine | R | 0.4 |
| Morphine | SC | 1 |
| Oxycodone | PO | 0.63 |
| Oxycodone | SC | 1.5 |
| Propoxyphene | IM | 0.167 |
| Propoxyphene | IV | 0.167 |
| Propoxyphene | PO | 0.08 |
| Propoxyphene | R | 0.08 |
| Propoxyphene | SC | 0.167 |
| Propoxyphene | TD | 0.167 |
| Sufentanil | SC | 1 |
| Sufentanil | IV | 1 |
| Sufentanil | PO | 0.5 |
| Sufentanil | SL | 0.5 |
| Tramadol | PO | 0.05 |
| Tramadol | SC | 0.1 |

Note:
1. MEDD calculation: [DOSE] × [MEDD_FACTOR]
2. Abbreviation list

| | |
|---|---|
| PO | Oral |
| IM | Intramuscular |
| IV | Intravenous |
| SC | Subcutaneous |
| SL | Sublingual |
| R | Rectal |
| EP | Epidural |
| IT | Intrathecal |
| TD | Transdermal |

Table 1 is used in the following manner to calculate equivalent doses of morphine and other analgesics administered by various routes. For a particular opioid A administered by route α, the equivalent dose of opioid B administered by route β is calculated as:

Dose of opioid B=dose of opioid A×(MEDD-factor for opioid A administered by route α)/(MEDD-factor for opioid B administered by route β).

For examples, the MEDD-factor for morphine administered orally is 0.4, whereas the MEDD-factor for morphine administered intravenously is 1.0. Thus, the dose of morphine administered intravenously that is equivalent to a 100 mg dose of morphine administered orally is calculated as 100 mg×(MEDD-Factor morphine PO)/(MEDD-Factor morphine IV)=100 mg×0.4/1.0=40 mg.

Similarly, the MEDD-factor for oxycodone administered orally is 0.63. Thus the dose of orally-administered oxycodone that is equivalent to a 100 mg dose of morphine administered orally is calculated as 100 mg×(MEDD-factor morphine PO)/(MEDD-factor oxycodone PO)=100 mg×0.4/0.63=63 mg.

The FDC also comprises flupirtine or retigabine in an amount that has a euphoria inhibiting effect and provides an adverse experience in the subject who takes more than the recommended dose (for example, at least twice the recommended dose) or takes the FDC by a route other than as intended.

As used herein, the expression “a euphoria-inhibiting effect” includes the suppression, cloaking, masking or countering of the euphoria-inducing properties of opioids. This occurs when the subject more than the recommended dose of the FDC, resulting in the flupirtine or retigabine inducing sedation and/or adverse side-effects, such as nausea and/or vomiting.

The concentration of flupirtine used in the methods and compositions of the present invention ranges from 25 mg to 1000 mg.

The concentration of the retigabine used in the methods and compositions of the present invention ranges from 10 mg to 500 mg.

The concentration of flupirtine or retigabine required in the FDC varies depending upon which opioid is used and its euphoria-inducing capacity which, in turn, correlates with its abuse potential. One of skill in the art will recognise that there are many factors which modify the action of the active substances used in the FDC, including age, body weight, sex, diet, condition of the subject, time of administration, the rare and route of administration.

The euphoria-inhibiting effect of taking more than the recommended dose of the FDC means that there is a reduced incentive for a user to take the FDC at a dose higher than the recommended dose. The Diagnostic and Statistical Manual of Mental Disorders IV (DSM-IV) specifies that one or more of the following symptoms must occur within a 12-month period in order to meet the diagnostic criteria for “abuse” includes:
Interference with role fulfilment: The individual's use of opioids repeatedly interferes with the ability to fulfil obligations at work, home, or school.
Danger to self: The individual repeatedly uses opioids in situations in which it may be physically hazardous (while driving a car, for example).
Legal problems: The individual has recurrent opioid related legal problems (such as arrests for possession of narcotics).
Social problems: The individual continues to use opioids despite repeated interpersonal or relationship problems caused by or made worse by the use of opioids.

As used herein, abuse of the opioid to obtain a sense of euphoria is distinct from the development of tolerance or dependence to an opioid.

The DSM-IV specifies that three or more of the following symptoms must occur at any time during a 12-month period in order to meet diagnostic criteria for dependence:
Tolerance: The individual either has to use increasingly higher amounts of the drug over time in order to achieve the same drug effect or finds that the same amount of the drug has much less of an effect over time than before.
Withdrawal: The individual either experiences the characteristic abstinence syndrome (i.e., opioid-specific withdrawal) or the individual uses opioids or similar-acting drugs in order to avoid or relieve withdrawal symptoms.
Loss of control: The individual either repeatedly uses more opioids than planned or uses the opioids over longer periods of time than planned.
Inability to stop using: The individual has either unsuccessfully attempted to cut down or stop using the opioids or has a persistent desire to stop using.
Time: The individual spends a lot of time obtaining opioids, getting money to buy opioids, using opioids, being under the influence of opioids, and recovering from the effects of opioids.
Interference with activities: The individual either gives up or reduces the amount of time involved in recreational activities, social activities, and/or occupational activities.
Harm to self: The individual continues to use opioids despite having either a physical or psychological problem (depression, for example) that is caused or made worse by the opioid use.

One may evaluate the liability of an opioid-containing FDC product to be abused by conducting conditioned place conditioning experiments in animal models and/or product “liking” or preference experiments using human volunteers.

The term “pain” is intended to describe both acute and chronic pain, including nociceptive pain, inflammatory pain and neuropathic pain.

Neuropathic pain is often reported as having a lancinating or continuous burning character and is frequently associated with the appearance of abnormal sensory signs such as allodynia and hyperalgesia. Alloydnia is defined as pain resulting from a stimulus that does not normally elicit a painful response, and hyperalgesia is characterized by an increased pain response to normally non-painful stimuli. Some disorders characterized by neuropathic pain include monoradiculopathies, trigeminal neuralgia, postherpetic neuralgia, phantom limb pain, complex regional pain syndromes, back pain and the various peripheral neuropathies. Neuropathic pain may also be associated with diabetes, radio- or chemo-therapy and infections such as HIV. Neuropathic pain may also result as a side effect of drug treatment or abuse.

For clinical purposes, nociceptive pain can be classified as somatic or visceral. Somatic pain results from prolonged activation of nociceptive receptors in somatic tissues such as a bone, joint, muscle or skin. Visceral pain, on the other hand manifests from activation of nociceptive receptors by pathological mechanisms such as mechanical injury, x-ray irradiation and toxic agents.

Neuropathic pain can be characterized by the following clinical features (Teng and Mekhail Pain Practice 3:8-12, 2003, Rajbhandari et al. Pain 83:627-629, 1999, Melzack et al. Ann NY Acad Sci, 933: 157-174, 2001):
1. There is the presence of an abnormal, unpleasant sensation (dysesthesia) that frequently has a burning or electrical quality with an occasional paroxysmal, brief, shooting, or stabbing quality.
2. Although the onset of most neuropathic pain is within days after the precipitating injury, there is no absolute temporal relationship to the originating neural trauma such that it can begin weeks, months, or even years later.
3. Pain may be felt in a region of sensory deficit.
4. Non-noxious stimuli may be painful (allodynia).
5. Noxious stimuli may produce greater than normal response (hyperalgesia).
6. There may be an increase in the intensity of pain with repeated stimuli and the pain may persist after the removal of stimuli.

Throughout this specification, the term “neuropathic pain” is to be understood to mean pain initiated or caused by a primary lesion or dysfunction within the nervous system. Examples of categories of neuropathic pain that may be treated by the methods of the present invention include monoradiculopathies, trigeminal neuralgia, postherpetic neuralgia, phantom limb pain, complex regional pain syndromes, back pain, neuropathic pain associated with AIDS and infection with the human immunodeficiency virus and the various peripheral neuropathies, including, but not limited to drug-induced and diabetic neuropathies.

In a further embodiment, the present invention extends to treating pain associated with any one or more of the following diseases which cause neuropathic pain or which have a neuropathic pain component: abdominal wall defect, abdominal migraine, achondrogenesis, achondrogenesis Type IV, achondrogenesis Type III, achondroplasia, achondroplasia tarda, achondroplastic dwarfism, acquired humanimmunodeficiency syndrome (AIDS), acute intermittent porphyria, acute porphyrias, acute shoulder neuritis, acute toxic epidermolysis, adiposa dolorosa, adrenal neoplasm, adrenomyeloneuropathy, adult dermatomyositis, amyotrophic lateral sclerosis, amyotrophic lateral sclerosis-polyglucosan bodies, AN, AN 1, AN 2, anal rectal malformations, anal stenosis, arachnitis, arachnoiditis ossificans, arachnoiditis, arteritis giant cell, arthritis, arthritis urethritica, ascending paralysis, astrocytoma grade I (Benign), astrocytoma grade II (Benign), athetoid cerebral palsy, Barrett esophagus, Barrett ulcer, benign tumors of the central nervous system, bone tumor-epidermoid cyst-polyposis, brachial neuritis, brachial neuritis syndrome, brachial plexus neuritis, brachial-plexus-neuropathy, brachiocephalic ischemia, brain tumors, brain tumors benign, brain tumors malignant, brittle bone disease, bullosa hereditaria, bullous cie, bullous congenital ichthyosiform erythroderma, bullous ichthyosis, bullous pemphigoid, Burkitt's lymphoma, Burkitt's lymphoma African type, Burkitt's lymphoma non-African type, calcaneal valgus, calcaneovalgus, cavernous lymphangioma, cavernous malformations, central form neurofibromatosis, cervical spinal stenosis, cervical vertebral fusion, Charcot's disease, Charcot-Marie-Tooth disease, Charcot-Marie-Tooth disease variant, Charcot-Marie-Tooth-Roussy-Levy disease, childhood dermatomyositis, chondrodysplasia punctata, chondrodystrophia calcificans congenita, chondrodystrophia fetalis, chondrodystrophic myotonia, chondrodystrophy, chondrodystrophy with clubfeet, chondrodystrophy epiphyseal, chondrodystrophy hyperplastic form, chondroectodermal dysplasias, chondrogenesis imperfecta, chondrohystrophia, chondroosteodystrophy, chronic adhesive arachnoiditis, chronic idiopathic polyneuritis (CIP), chronic inflammatory demyelinating polyneuropathy, chronic inflammatory demyelinating polyradiculoneuropathy, cicatricial pemphigoid, complex regional pain syndrome, congenital cervical synostosis, congenital dysmyelinating neuropathy, congenital hypomyelinating polyneuropathy, congenital hypomyelination neuropathy, congenital hypomyelination, congenital hypomyelination (onion bulb) polyneuropathy, congenital ichthyosiform erythroderma, congenital tethered cervical spinal cord syndrome, cranial arteritis, Crohn's disease, cutaneous porphyrias, degenerative lumbar spinal stenosis, demyelinating disease, diabetes mellitus diabetes insulin dependent, diabetes mellitus, diabetes mellitus Addison's disease myxedema, discoid lupus, discoid lupus erythematosus, disseminated lupus erythematosus, disseminated neurodermatitis, disseminated sclerosis, eds kyphoscoliotic, eds kyphoscoliosis, eds mitis type, eds ocular-scoliotic, elastosis dystrophica syndrome, encephalofacial angiomatosis, encephalotrigeminal angiomatosis, enchondromatosis with multiple cavernous hemangiomas, endemic polyneuritis, endometriosis, eosinophilic fasciitis, epidermolysis bullosa, epidermolysis bullosa acquisita, epidermolysis bullosa hereditaria, epidermolysis bullosa letalias, epidermolysis hereditaria tarda, epidermolytic hyperkeratosis, epidermolytic hyperkeratosis, familial lumbar stenosis, familial lymphedema praecox, fibromyalgia, fibromyalgia-fibromyositis, fibromyositis, fibrositis, fibrous ankylosis of multiple joints, fibrous dysplasia, fragile x syndrome, generalized fibromatosis, guillain-barre syndrome, hemangiomatosis chondrodystrophica, hereditary sensory and autonomic neuropathy type I, hereditary sensory and autonomic neuropathy type II, hereditary sensory and autonomic neuropathy type III, hereditary sensory motor neuropathy, hereditary sensory neuropathy type I hereditary sensory neuropathy type i, hereditary sensory neuropathy type II, hereditary sensory neuropathy type M, hereditary sensory radicular neuropathy type I, hereditary sensory radicular neuropathy type I, hereditary sensory radicular neuropathy type II, herpes zoster, Hodgkin disease, Hodgkin's disease, Hodgkin's lymphoma, hyperplastic epidermolysis bullosa, hypertrophic interstitial neuropathy, hypertrophic interstitial neuritis, hypertrophic interstitial radiculoneuropathy, hypertrophic neuropathy of refsum, idiopathic brachial plexus neuropathy, idiopathic cervical dystonia, juvenile (childhood) dermatomyositis (jdms), juvenile diabetes, juvenile rheumatoid arthritis, pes planus, leg ulcer, lumbar canal stenosis, lumbar spinal stenosis, lumbosacral spinal stenosis, lupus, lupus, lupus erythematosus, lymphangiomas, mononeuritis multiplex, mononeuritis peripheral, mononeuropathy peripheral, monostotic fibrous dysplasia, multiple cartilaginous enchondroses, multiple cartilaginous exostoses, multiple enchondromatosis, multiple myeloma, multiple neuritis of the shoulder girdle, multiple osteochondromatosis, multiple peripheral neuritis, multiple sclerosis, musculoskeletal pain syndrome, neuropathic amyloidosis, neuropathic beriberi, neuropathy of brachialpelxus syndrome, neuropathy hereditary sensory type I, neuropathy hereditary sensory type II, Nieman pick disease type a (acute neuronopathic form), Nieman pick disease type b, Nieman pick disease type c (chronic neuronopathic form), non-scarring epidermolysis bullosa, ochronotic arthritis, ocular herpes, onion-bulb neuropathy, osteogenesis imperfect, osteogenesis imperfecta, osteogenesis imperfecta congenita, osteogenesis imperfecta tarda, peripheral neuritis, peripheral neuropathy, perthes disease, polyarteritis nodosa, polymyalgia rheumatica, polymyositis and dermatomyositis, polyneuritis peripheral, polyneuropathy peripheral, polyneuropathy and polyradiculoneuropathy, polyostotic fibrous dysplasia, polyostotic sclerosing histiocytosis, postmyelographic arachnoiditis, primary progressive multiple sclerosis, psoriasis, radial nerve palsy, radicular neuropathy sensory, radicular neuropathy sensory recessive, reflex sympathetic dystrophy syndrome, relapsing-remitting multiple sclerosis, sensory neuropathy hereditary type I, sensory neuropathy hereditary type II, sensory neuropathy hereditary type I, sensory radicular neuropathy, sensory radicular neuropathy recessive, sickle cell anemia, sickle cell disease, sickle cell-hemoglobin c disease, sickle cell-hemoglobin d disease, sickle cell-thalassemia disease, sickle cell trait, spina bifida, spina bifida aperta, spinal arachnoiditis, spinal arteriovenous malformation, spinal ossifying arachnoiditis, spinal stenosis, stenosis of the lumbar vertebral canal, still's disease, syringomyelia, systemic sclerosis, talipes calcaneus, talipes equinovarus, talipes equinus, talipes varus, talipes valgus, tandem spinal stenosis, temporal arteritis/giant cell arteritis, temporal arteritis, tethered spinal cord syndrome, tethered cord malformation sequence, tethered cord syndrome, tethered cervical spinal cord syndrome, thalamic pain syndrome, thalamic hyperesthetic anesthesia, trigeminal neuralgia, variegate porphyria, vertebral ankylosing hyperostosis amongst others.

The term “inflammatory pain” or a pain associated with inflammation is intended to describe the subset of acute and chronic pain that results from inflammatory processes, such as may arise in the case of infections, arthritis and neoplasia or tumor related hypertrophy. Inflammatory pain includes pain associated with rheumatoid arthritis, osteo-arthritis, psoriatic arthropathy, arthritis associated with other inflammatory and autoimmune conditions, degenerative conditions such as back strain and mechanical back pain or disc disease, post operative pain, pain from an injury such as a soft tissue bruise or strained ligament or broken bone, abscess or cellulitis, fibrositis or myositis. Examples of inflammatory conditions include, but are not limited to, inflammatory diseases and disorders which result in a response of redness, swelling, pain, and a feeling of heat in certain areas that is meant to protect tissues affected by injury or disease. Inflammatory diseases which include a pain component which can be relieved using the compositions and methods of the present invention include, without being limited to, acne, angina, arthritis, aspiration pneumonia, disease, empyema, gastroenteritis, inflammation, intestinal flu, NEC, necrotizing enterocolitis, pelvic inflammatory disease, pharyngitis, PID, pleurisy, raw throat, redness, rubor, sore throat, stomach flu and urinary tract infections, chronic inflammatory demyelinating polyneuropathy, chronic inflammatory demyelinating Polyradiculoneuropathy, chronic inflammatory demyelinating polyneuropathy, chronic inflammatory demyelinating polyradiculoneuropathy.

In a further embodiment, the present invention provides methods and compositions for alleviating the pain associated with cancer.

In one particular embodiment, the FDC comprising an opioid and flupirtine or retigabine are used during or following cancer treatment. Examples of cancers which contain a pain component which may be relieved using the compositions and methods of the present invention include but are not limited to abl1 protooncogene, aids related cancers, acoustic neuroma, acute lymphocytic leukaemia, acute myeloid leukaemia, adenocystic carcinoma, adrenocortical cancer, agnogenic myeloid metaplasia, alopecia, alveolar soft-part sarcoma, anal cancer, angiosarcoma, aplastic anaemia, astrocytoma, ataxia-telangiectasia, basal cell carcinoma (skin), bladder cancer, bone cancers, bowel cancer, brain stem glioma, brain and CNS tumors, breast cancer, CNS tumors, carcinoid tumors, cervical cancer, childhood brain tumors, childhood cancer, childhood leukaemia, childhood soft tissue sarcoma, chondrosarcoma, choriocarcinoma, chronic lymphocytic leukaemia, chronic myeloid leukaemia, colorectal cancers, cutaneous T-cell lymphoma, dermatofibrosarcoma-protuberans, desmoplastic-small-round-cell-tumor, ductal carcinoma, endocrine cancers, endometrial cancer, ependymoma, esophageal cancer, Ewing's sarcoma, extra-hepatic bile duct cancer, eye cancer, eye: melanoma, retinoblastoma, fallopian tube cancer, fanconi anaemia, fibrosarcoma, gall bladder cancer, gastric cancer, gastrointestinal cancers, gastrointestinal-carcinoid-tumor, genitourinary cancers, germ cell tumors, gestational-trophoblastic-disease, glioma, gynaecological cancers, haematological malignancies, hairy cell leukaemia, head and neck cancer, hepatocellular cancer, hereditary breast cancer, histiocytosis, Hodgkin's disease, human papillomavirus, hydatidiform mole, hypercalcemia, hypopharynx cancer, intraocular melanoma, islet cell cancer, Kaposi's sarcoma, kidney cancer, Langerhan's-cell-histiocytosis, laryngeal cancer, leiomyosarcoma, leukaemia, Li-fraumeni syndrome, lip cancer, liposarcoma, liver cancer, lung cancer, lymphedema, lymphoma, Hodgkin's lymphoma, non-Hodgkin's lymphoma, male breast cancer, malignant-rhabdoid-tumor-of-kidney, medulloblastoma, melanoma, merkel cell cancer, mesothelioma, metastatic cancer, mouth cancer, multiple endocrine neoplasia, mycosis fungoides, myelodysplastic syndromes, myeloma, myeloproliferative disorders, nasal cancer, nasopharyngeal cancer, nephroblastoma, neuroblastoma, neurofibromatosis, nijmegen breakage syndrome, non-melanoma skin cancer, non-small-cell-lung-cancer-(NSCLC), ocular cancers, oesophageal cancer, oral cavity cancer, oropharynx cancer, osteosarcoma, ostomy ovarian cancer, pancreas cancer, paranasal cancer, parathyroid cancer, parotid gland cancer, penile cancer, peripheral-neuroectodermal-tumors, pituitary cancer, polycythemia vera, prostate cancer, rare-cancers-and-associated-disorders, renal cell carcinoma, retinoblastoma, rhabdomyosarcoma, Rothmund-Thomson syndrome, salivary gland cancer, sarcoma, schwannoma, Sezary syndrome, skin cancer, small cell lung cancer (SCLC), small intestine cancer, soft tissue sarcoma, spinal cord tumors, squamous-cell-carcinoma-(skin), stomach cancer, synovial sarcoma, testicular cancer, thymus cancer, thyroid cancer, transitional-cell-cancer-(bladder), transitional-cell-cancer-(renal-pelvis−/−ureter), trophoblastic cancer, urethral cancer, urinary system cancer, uroplakins, uterine sarcoma, uterus cancer, vaginal cancer, vulva cancer, Waldenstrom's-macroglobulinemia or Wilms' tumor.

The method according to the present invention to induces an analgesic response to neuropathic and/or inflammatory pain being suffered by a subject, whilst reducing the enticement for substance abuse. A subject, in this context, is also referred to as a “patient”, “target” or “recipient”. In this context the terms “analgesia” and “analgesic response” are intended to describe a state of reduced sensibility to pain, which occurs without overt sedation and without an effect upon the sense of touch. In one aspect, the sensibility to pain is reduced by at least 30%, at least 50%, at least 70% and or at least 85%. In another aspect of the present invention, the sensibility to the pain is completely, or substantially completely, removed. To assess the level of reduction of sensibility to pain associated with the analgesia induced by the methods according to the present invention it is possible to conduct tests such as the short form McGill pain questionnaire and/or visual analogue scales for pain intensity and/or verbal rating scales for pain intensity and/or measurement of tactile allodynia using von Frey hairs or similar device. These tests are standard tests within the art and would be well known to the skilled person.

In a related aspect, the FDC of the present invention results in an analgesic response without inducing overt sedation, whilst reducing the enticement for substance abuse.

By the term “overt sedation” it is intended to convey that the compositions described herein, when used at the recommended dose, do not result in a level of sedation of the patient or subject being treated which shows significant, visible or apparent drowsiness or cause unconsciousness of the patient being treated. Thus, the treatment methods and compositions herein, in one embodiment, do not result in sleepiness or drowsiness in the patient that interfere with, or inhibit, the activities associated with day to day living, such as driving a motor vehicle or operating machinery for human subjects, or feeding and grooming for animal subjects. The term “without overt sedation” also means inducing an analgesic effect without causing significant cognitive or general impairment of nervous system function (such as attentiveness or wakefulness). Such effects on cognition can lead to a change in the measurement that leads to an erroneous conclusion about the level or type of pain or effect of amelioration of symptoms.

The active agents may be administered for therapy by any suitable route. It will be understood that the active agents are administered in one embodiment via a route that does not result in overt sedation of the subject when used at the recommended dose, or result in dose-limiting side effects. Suitable routes of administration may include oral, rectal, nasal, inhalation of aerosols or particulates, topical (including buccal and sublingual), transdermal except when administered in combination with a safe skin-tolerant sunscreen enhancer and at least one volatile liquid), vaginal, intravesical, parenteral (including subcutaneous, intramuscular, intravenous, intrasternal, intra-articular, injections into the joint, and intradermal) and intrathecal or epidural. In one embodiment, administration of the active agent is by a route resulting in first presentation of the compound to the stomach of the subject. In this embodiment, the active agents are generally administered via an oral route. In another embodiment the active agents are administered by the transdermal route. However, it will be appreciated that the route may vary with the condition and age of the subject, the nature of the pain being treated, its location within the subject and the judgement of the physician or veterinarian. It will also be understood that individual active agents may be administered by the same or different distinct routes. The individual active agents may be administered separately or together directly into a joint involved with an inflammatory painful process.

The FDC may be administered to a subject at a rate that delivers between 1 and 200 mg oral morphine per day to a patient, or equivalent doses of morphine delivered by alternative routes, or equivalent doses of an opioid other than morphine delivered orally or by alternative routes, as determined from a table of MEDD factors for morphine and other opioid drugs such as Table 1.

Accordingly, a treatment protocol is contemplated for treating pain in a subject, the protocol comprising the steps of administration to the subject an effective amount of an abuse-deterrent FDC comprising an opioid and flupirtine or retigabine.

A further aspect also provides an abuse-deterrent composition comprising an opioid and flupirtine or retigabine together with one or more pharmaceutically acceptable additives and optionally other medicaments. The pharmaceutically acceptable additives may be in the form of carriers, diluents, adjuvants and/or excipients and they include all conventional solvents, dispersion agents, fillers, solid carriers, coating agents, antifungal or antibacterial agents, dermal penetration agents, surfactants, isotonic and absorption agents and slow or controlled release matrices. The active agents may be presented in the form of a kit of components adapted for allowing concurrent, separate or sequential administration of the active agents. Each carrier, diluent, adjuvant and/or excipient must be “pharmaceutically acceptable” in the sense of being compatible with the other ingredients of the composition and physiologically tolerated by the subject. The compositions may conveniently be presented in unit dosage form and may be prepared by methods well known in the art of pharmacy. Such methods include the step of bringing into association the active ingredient with the carrier, which constitutes one or more accessory ingredients. In general, the compositions are prepared by uniformly and intimately bringing into association the active ingredient with liquid carriers, diluents, adjuvants and/or excipients or finely divided solid carriers or both, and then if necessary shaping the product.

Compositions of the present invention suitable for oral administration may be presented as discrete units such as capsules, sachets or tablets each containing a predetermined amount of the active ingredient; as a powder or granules; as a solution or a suspension in an aqueous phase or non-aqueous liquid; or as an oil-in-water liquid emulsion or a water-in-oil emulsion. The active ingredient may also be presented as a bolus, electuary or paste.

A tablet may be made by compression or moulding, optionally with one or more accessory ingredients. Compressed tablets may be prepared by compressing in a suitable machine the active ingredient in a free-flowing form such as a powder or granules, optionally mixed with a binder (e.g. inert diluent, preservative disintegrant, sodium starch glycollate, cross-linked povidone, cross-linked sodium carboxymethyl cellulose) surface-active or dispersing agent. Moulded tablets may be made my moulding in a suitable machine a mixture of the powdered compound moistened with an inert liquid diluent. The tablets may optionally be coated or scored and may be formulated so as to provide slow or controlled release of the active ingredient therein using, for example, hydroxypropylmethyl cellulose in varying proportions to provide the desired release profile. Tablets may optionally be provided with an enteric coating, to provide release in parts of the gut other than the stomach.

Compositions suitable for parenteral administration include aqueous and non-aqueous isotonic sterile injection solutions which may contain anti-oxidants, buffers, bacteriostats and solutes which render the composition isotonic with the blood of the intended subject; and aqueous and non-aqueous sterile suspensions which may include suspended agents and thickening agents. The compositions may be presented in a unit-dose or multi-dose sealed containers, for example, ampoules and vials, and may be stored in a freeze-dried (lyophilized) condition requiring only the addition of the sterile liquid carrier, for example water for injections, immediately prior to use. Extemporaneous injection solutions and suspensions may be prepared from sterile powders, granules and tablets of the kind previously described. When reconstituted these can be in the form of aqueous solution, dissolved in water, isotonic saline or a balanced salt solution. Additionally, when reconstituted the product could be a suspension in which the compound(s) is/are dispersed in the liquid medium by combination with liposomes or a lipid emulsion such as soya bean.

Compositions suitable for topical administration to the skin, i.e. transdermal administration (except when administered in combination with a safe skin-tolerant sunscreen enhancer and at least one volatile liquid), may comprise the active agents dissolved or suspended in any suitable carrier or base and may be in the form of lotions, gels, creams, pastes, ointments and the like. Suitable carriers may include mineral oil, propylene glycol, waxes, polyoxyethylene and long chain alcohols. Transdermal devices, such as patches may also be used and may comprise a microporous membrane made from suitable material such as cellulose nitrate/acetate, propylene and polycarbonates. The patches may also contain suitable skin adhesive and backing materials.

The active compounds described herein may also be presented as implants, which may comprise a drug bearing polymeric device wherein the polymer is biocompatible and non-toxic. Suitable polymers may include hydrogels, silicones, polyethylenes and biodegradable polymers.

The compounds of the subject invention may be administered in a sustained (i.e. controlled) or slow release form. A sustained release preparation is one in which the active ingredient is slowly released within the body of the subject once administered and maintains the desired drug concentration over a minimum period of time. The preparation of sustained release formulations is well understood by persons skilled in the art. Dosage forms may include oral forms, implants and transdermal forms, joint injections, sustained or slow release injectables. For slow release administration, the active ingredients may be suspended as slow release particles or within liposomes, for example.

The compositions herein may be packaged for sale with other active agents or alternatively, other active agents may be formulated with flupirtine or retigabine or their pharmaceutical salts thereof and an opioid. The composition may be sold or provided with a set of instructions in the form of a therapeutic protocol. This protocol may also include, in one embodiment, a selection process for type of patient or type of condition or a type of pain such as a nociceptive component of inflammatory or neuropathic pain.

The FDC may provide an immediate release form of the opioid and the flupirtine or retigabine. In a further aspect, the FDC provides a sustained release form of the opioid, and provides part or all of the flupirtine or retigabine in (i) immediate release form; (ii) sustained release form, or (iii) both immediate and sustained release form, in combination with part of all of the opioid in sustained release form. Sustained release may be accomplished in accordance with formulations/methods of manufacture known to those of skill in the art e.g., via the incorporation of the opioid and the flupirtine or retigabine in a controlled release carrier; or via a controlled release coating of a carrier containing the opioid and/or the flupirtine or retigabine.

In one aspect, the FDC comprises a sustained release carrier. Alternatively, a normal release carrier having a coating that controls the release of the flupirtine or retigabine and/or the opioid can be used. Suitable base materials for controlled release carriers include combinations of higher aliphatic alcohols and acrylic resins.

Base compositions prepared from such higher aliphatic alcohols and acrylic resins provide sustained release of the opioid and/or the flupirtine or retigabine over a period of time, for example, from about 1 hour to about 24 hours.

A pharmaceutically acceptable acrylic polymer can be used in the methods and compositions of the present invention. The acrylic polymers may be cationic, anionic, or non-ionic polymers and may be acrylates, methacrylates, formed of methacrylic acid or methacrylic acid esters. These polymers can be synthesised to be cationic, anionic or non-ionic, which renders the polymers that would be pH dependent and consequently soluble in, or resistant to solutions over a wide range of pHs.

In addition, suitable materials for inclusion in a controlled release carrier include:
(a) hydrophilic polymers, such as, gums, cellulose ethers, acrylic resins and protein derived materials. Examples of specific polymers include, without being limited to, hydroxyalkylcellulose and carboxyalkylcellulose.
(b) digestible, long chain (C₈-C₅₀, in particular C₁₂-C₄₀) substituted or unsubstituted hydrocarbons, such as fatty acids, fatty alcohols, glycerol esters or fatty acids, mineral and vegetable oils and waxes.
(c) polyalkylene glycols.

One particularly suitable carrier comprises at least one water soluble hydroxyalkyl cellulose, at least one C12-C36, preferably C14-C22, aliphatic alcohol and, optionally, at least one polyalkylene glycol.

The at least one hydroxyalkyl cellulose is preferably a hydroxy (C1 to C6) alkyl cellulose, such as hydroxypropylcellulose, hydroxypropylmethylcellulose and, especially, hydroxyethyl cellulose. The amount of the at least one hydroxyalkyl cellulose in the present pharmaceutical dosage form will be determined, inter alia, by the precise rate of opioid analgesic release required. Preferably however, the oral dosage form contains between 1% and 45%, especially between 5% and 25% (by weight) of the at least one hydroxyalkyl cellulose.

While the at least one aliphatic alcohol may be, for example, lauryl alcohol, myristyl alcohol or stearyl alcohol, in particularly preferred embodiments the at least one aliphatic alcohol is cetyl alcohol or cetostearyl alcohol. The amount of the at least one aliphatic alcohol in the present dosage form will be determined, as above, by the precise rate of opioid analgesic release required. It will also depend on whether at least one polyalkylene glycol is present in or absent from the dosage form. In the absence of at least one polyalkylene glycol, the dosage form preferably contains between 20% and 50% (by weight) of the at least one aliphatic alcohol. When at least one polyalkylene glycol is present in the dosage form, then the combined weight of the at least one aliphatic alcohol and the at least one polyalkylene glycol preferably constitutes between 20% and 50% (by weight) of the total dosage.

In the present preferred dosage form, the ratio of, e.g., the at least one hydroxyalkyl cellulose or acrylic resin to the at least one aliphatic alcohol/polyalkylene glycol determines, to a considerable extent, the release rate of the opioid analgesic from the formulation. A ratio of the at least one hydroxyalkyl cellulose to the at least one aliphatic alcohol/polyalkylene glycol of between 1:2 and 1:4 is preferred, with a ratio of between 1:3 and 1:4 being particularly preferred.

The at least one polyalkylene glycol may be, for example, polypropylene glycol or polyethylene glycol, which is preferred. The number average molecular weight of the at least one polyalkylene glycol is preferred between 1000 and 15000 especially between 1500 and 12000.

Another suitable controlled release carrier would comprise an alkylcellulose (especially ethyl cellulose), a C12 to C36 aliphatic alcohol and, optionally, a polyalkylene glycol. In addition to the above ingredients, a controlled release carrier may also contain suitable quantities of other materials, e.g. diluents, lubricants, binders, granulating aids, colorants, flavorants and glidants that are conventional in the pharmaceutical art.

As an alternative to a controlled release carrier, the present carrier may be a normal release carrier having a coat that controls the release of the drug. In particularly preferred embodiments of this aspect of the invention, the present dosage form comprises film coated spheroids containing active ingredient and a non-water soluble spheronising agent. The term spheroid is known in the pharmaceutical art and means a spherical granule having a diameter of between 0.5 mm and 2.5 mm especially between 0.5 mm and 2 mm.

The spheronising agent may be any pharmaceutically acceptable material that, together with the active ingredient, can be spheronised to form spheroids.

Microcrystalline cellulose is preferred. According to a preferred aspect of the present invention, the film coated spheroids contain between 70% and 99% (by wt), especially between 80% and 95% (by wt), of the spheronising agent, especially microcrystalline cellulose.

In addition to the active ingredient and spheronising agent, the spheroids may also contain a binder. Suitable binders, such as low viscosity, water soluble polymers, will be well known to those skilled in the pharmaceutical art. However, water soluble hydroxy lower alkyl cellulose, such as hydroxy propyl cellulose, are preferred. Additionally (or alternatively) the spheroids may contain a water insoluble polymer, especially an acrylic polymer, an acrylic copolymer, such as a methacrylic acid-ethyl acrylate copolymer, or ethyl cellulose.

The spheroids are preferably film coated with a material that permits release of the opioid analgesic at a controlled rate in an aqueous medium. The film coat is chosen so as to achieve, in combination with the other ingredients, the in-vitro release rate outlined above (between 12.5% and 42.5% (by weight) release after 1 hour, etc.).

The film coat will generally include a water insoluble material such as: (a) a wax, either alone or in admixture with a fatty alcohol; (b) shellac or zein; (c) a water insoluble cellulose, especially ethyl cellulose; (d) a polymethacrylate.

Preferably, the film coat comprises a mixture of the water insoluble material and a water soluble material. The ratio of water insoluble to water soluble material is determined by, amongst other factors, the release rate required and the solubility characteristics of the materials selected.

The water soluble material may be, for example, polyvinylpyrrolidone or, which is preferred, a water soluble cellulose, especially hydroxypropylmethyl cellulose.

Suitable combinations of water insoluble and water soluble materials for the film coat include shellac and polyvinylpyrrolidone or, which is preferred, ethyl cellulose and hydroxypropylmethyl cellulose.

In another embodiment, in order to obtain a sustained-release of the opioid sufficient to provide an analgesic effect for the extended durations set forth in the present invention, the substrate comprising the therapeutically active agent may be coated with a sufficient amount of hydrophobic material to obtain a weight gain level from about 2 to about 30 percent, although the overcoat may be greater depending upon the physical properties of the particular opioid analgesic compound utilized and the desired release rate, among other things.

The solvent which is used for the hydrophobic material may be any pharmaceutically acceptable solvent, including water, methanol, ethanol, methylene chloride and mixtures thereof. It is preferable however, that the coatings be based upon aqueous dispersions of the hydrophobic material.

In certain embodiments of the present invention, the hydrophobic polymer comprising the sustained-release coating is a pharmaceutically acceptable acrylic polymer, including but not limited to acrylic acid and methacrylic acid copolymers, methacrylic acid copolymers, methyl methacrylate copolymers, ethoxyethyl methacrylates, cynaoethyl methacrylate, methyl methacrylate, copolymers, methacrylic acid copolymers, methyl methacrylate copolymers, methyl methacrylate copolymers, methyl methacrylate copolymers, methacrylic acid copolymer, aminoalkyl methacrylate copolymer, methacrylic acid copolymers, methyl methacrylate copolymers, poly (acrylic acid), poly (methacrylic acid, methacrylic acid alkylamide copolymer, poly (methyl methacrylate), poly (methacrylic acid) (anhydride), methyl methacrylate, polymethacrylate, methyl methacrylate copolymer, poly (methyl methacrylate), poly (methyl methacrylate) copolymer, polyacrylamide, aminoalkyl methacrylate copolymer, poly (methacrylic acid anhydride), and glycidyl methacrylate copolymers.

In other embodiments, the hydrophobic polymer which may be used for coating the substrates of the present invention is a hydrophobic cellulosic material such as ethylcellulose. Those skilled in the art will appreciate that other cellulosic polymers, including other alkyl cellulosic polymers, may be substituted for part or all of the ethylcellulose included in the hydrophobic polymer coatings of the present invention.

In embodiments of the present invention where the coating comprises an aqueous dispersion of a hydrophobic polymer, the inclusion of an effective amount of a plasticizer in the aqueous dispersion of hydrophobic polymer will further improve the physical properties of the film. For example, because ethylcellulose has a relatively high glass transition temperature and does not form flexible films under normal coating conditions, it is necessary to plasticize the ethylcellulose before using the same as a coating material.

Generally, the amount of plasticizer included in a coating solution is based on the concentration of the film-former, e.g., most often from about 1 to about 50 percent by weight of the film-former. Concentration of the plasticizer, however, can only be properly determined after careful experimentation with the particular coating solution and method of application.

Examples of suitable plasticizers for ethylcellulose include water insoluble plasticizers such as dibutyl sebacate, diethyl phthalate, triethyl citrate, tributyl citrate, and triacetin, although it is possible that other water-insoluble plasticizers (such as acetylated monoglycerides, phthalate esters, castor oil, etc.) may be used. Triethyl citrate is especially preferred.

Examples of suitable plasticizers for the acrylic polymers of the present invention include citric acid esters such as triethyl citrate NF XVI, tributyl citrate, dibutyl phthalate, and possibly 1,2-propylene glycol, polyethylene glycols, propylene glycol, diethyl phthalate, castor oil, and triacetin, although it is possible that other water-insoluble plasticizers (such as acetylated monoglycerides, phthalate esters, castor oil, etc.) may be used. Triethyl citrate is especially preferred.

The sustained-release profile of the formulations of the invention can be altered, for example, by varying the thickness of the hydrophobic coating, changing the particular hydrophobic material used, or altering the relative amounts of, e.g., different acrylic resin lacquers, altering the manner in which the plasticizer is added (e.g., when the sustained-release coating is derived from an aqueous dispersion of hydrophobic polymer), by varying the amount of plasticizer relative to hydrophobic polymer, by the inclusion of additional ingredients or excipients, by altering the method of manufacture, etc.

Sustained-release spheroids or beads, coated with a therapeutically active agent are prepared, e.g. by dissolving the opioid analgesic in water and then spraying the solution onto a substrate using a Wurster insert. Optionally, additional ingredients are also added prior to coating the beads in order to assist the opioid analgesic binding to the substrates, and/or to color the solution, etc. For example, a product which includes hydroxypropyl methylcellulose, etc. with or without colorant may be added to the solution and the solution mixed (e.g., for about 1 hour) prior to application of the same onto the beads. The resultant coated substrate, in this example beads, may then be optionally overcoated with a barrier agent, to separate the therapeutically active agent from the hydrophobic sustained-release coating. An example of a suitable barrier agent is one which comprises hydroxypropyl methylcellulose. However, any film-former known in the art may be used. It is preferred that the barrier agent does not affect the dissolution rate of the final product.

The coating solutions of the present invention may contain, in addition to the film-former, plasticizer, and solvent system (i.e., water), a colorant to provide elegance and product distinction. Color may be added to the solution of the therapeutically active agent instead, or in addition to the aqueous dispersion of hydrophobic polymer.

The plasticized aqueous dispersion of hydrophobic polymer may be applied onto the substrate comprising the therapeutically active agent by spraying using any suitable spray equipment known in the art. In a preferred method, a Wurster fluidized-bed system is used in which an air jet, injected from underneath, fluidizes the core material and effects drying while the acrylic polymer coating is sprayed on. A sufficient amount of the aqueous dispersion of hydrophobic polymer to obtain a predetermined sustained-release of said therapeutically active agent when said coated substrate is exposed to aqueous solutions, e.g. gastric fluid, is preferably applied, taking into account the physically characteristics of the therapeutically active agent, the manner of incorporation of the plasticizer, etc. After coating with the hydrophobic polymer, a further overcoat of a film-former is optionally applied to the beads. This overcoat is provided, if at all, in order to substantially reduce agglomeration of the beads.

Next, the coated beads are cured in order to obtain a stabilized release rate of the therapeutically active agent.

In another embodiment, the pharmaceutical dosage form of the present invention is an aqueous suspension. Aqueous suspensions can contain the composition in admixture with pharmaceutically acceptable excipients such as suspending agents, e.g., sodium carboxymethyl cellulose, methylcellulose, hydroxypropylmethylcellulose, sodium alginate, polyvinylpyrrolidone, and natural gums such as gum tragacanth and gum acacia; dispersing or wetting agents such as naturally occurring phosphatide and lecithin, or condensation products of an alkylene oxide with fatty acids, e.g., polyoxyethylene stearate, or condensation products of ethylene oxide with long chain aliphatic alcohols, e.g., heptadecaethyleneoxycetanol, or condensation products of ethylene oxide with partial esters derived from fatty acids and a hexitol, e.g., polyoxyethylene sorbitol monoleate or condensation products of ethylene oxide with partial esters derived from fatty acids and hexitol anhydrides, e.g., polyoxyethylene sorbitan monooleate. Such aqueous suspensions can also contain one or more preservatives, e.g., ethyl- or n-propyl-p-hydroxy benzoate, one or more coloring agents, one or more flavoring agents and one or more sweetening agents, such as sucrose, saccharin or sodium or calcium cyclamate.

Dispersible powders and granules suitable for preparation of an aqueous suspension by the addition of water provide the composition in admixture with a dispersing of wetting agent, suspending agents and one or more preservatives. Suitable dispersing or wetting agents and suspending agents are exemplified by those already mentioned above. Additional excipients, e.g., sweetening, flavoring and coloring agents, can also be present. Syrups and elixirs can be formulated with sweetening agents, for example glycerol, sorbitol or sucrose. Such formulations can also contain a demulcent, a preservative and flavoring and coloring agents.

The FDC may be manufactured in a manner that makes it resistant to tampering or other behaviour that may otherwise counteract the abuse-deterrent properties of the FDC.

The present invention further contemplates a pain management protocol with a reduced enticement for substance abuse, said protocol comprising administering to a subject in need of pain management an amount of an opioid and an agent selected from flupirtine and retigabine which is effective to reduce the sensation of pain whilst reducing the enticement for substance abuse.

The present invention is now described with reference to the following non-limiting Examples.

### Example 1

### Animal Experiments to Demonstrate the Abuse Deterrent Properties of Analgesically Effective Fixed-Dose Combinations

### Rat Conditioned Place Preference Model

Conditioned place preference experiments are used to study drug seeking behaviour (e.g. Bardo and Bevins Psychopharmacology (Berl) 153:31-43, 2000, Hoffmann et al. Brain Research Bulletin 23:373-387, 1989). In this model, animals are trained to associate one particular environment (e.g. the interior of a dark chamber) with the administration of a particular drug or drug combination at a particular dose and another environment (e.g. the interior of a light chamber) with the administration of a reference drug or drug combination at a particular dose. After conditioning, animals are allowed unrestricted access to both environments on a drug-free test day. The relative amount of time spent in by an animal the environment in which the test drug or drug combination had previously been administered compared to the amount of time spent in the environment in which the reference drug or drug combination was administered reflects the animals preference for the test drug or drug combination compared to the reference drug or drug combination. With respect to FDCs containing an opioid drug and flupirtine or retigabine, the preferred outcome of such experiments is that the maximum non-sedating FDC is no more preferred by rats than a dose of the opioid drug alone equal to the dose of the opioid in the FDA, and no more preferred and preferably less preferred than a FDC that contains double the doses of opioid and flupirtine or retigabine determined to be a maximum non-sedating FDC.

Experiments are performed using a 2 chamber place preference apparatus (method described by Pelloux et al. Physiology Biochemistry and Behaviour 84:43-60, 2006), as shown below.

### Protocol

### 1. Preconditioning Period

the rats are allowed an initial period of unrestricted exploration of entire apparatus to determine unconditioned preference for A or B.

### 2. Conduct Place Preference Conditioning Using an Unbiased Procedure

each rat received one test drug or drug combination in one compartment, and a different test drug or drug combination in the other compartment. Experiments are conducted using the following conditions:
1. alternate pairings in successive days;
2. do 3 pairings each way (days 1-6);
3. assess place preference on day 7.

### Fixed Drug Combinations

### For Flupirtine/Morphine

Rats and humans may differ significantly in their responsiveness to particular drugs due to inter-species variations in metabolism, receptor density and receptor structure and function. Accordingly, the general operation of the invention is demonstrated in the rat model using as a reference doses of flupirtine and morphine administered alone and in combination that have been determines to confer analgesic efficacy without sedation.

Experiments using a Rotarod apparatus and a field activity monitor are conducted first to determine the maximum non-sedating doses and dose combinations of morphine and flupirtine. For young adult Wistar rats, intraperitoneal administration to 10 mg/kg flupirtine is a non-sedating dose, whereas 20 mg/kg flupirtine is sedating, according to the criteria of Goodchild et al. Pain Medicine 9:928-938, 2008. Intraperitoneal administration of 1.6 mg/kg morphine is not sedating. Intraperitoneal administration of an FDC containing 10 mg/kg flupirtine plus 1.6 mg/kg morphine is not sedating. Intraperitoneal administration of and FDC containing 20 mg/kg flupirtine and 3.2 mg/kg morphine is sedating.

Experiments may be performed using routes of administration other than intraperitoneal injection.

An intraperitoneally administered FDC containing 10 mg/kg flupirtine and 1.6 mg/kg morphine is effective as an analgesic in rat models of pain (Goodchild et al. 2008 supra).

The following FDC are tested are examined to show the efficacy of the model described in Example 1 in demonstrating opioid seeking/abuse tendencies of drugs alone and in combination:

| | | | |
|---|---|---|---|
| Morphine | 1.6 mg/kg | 3.2 mg/kg | 4.8 mg/kg |
| Flupirtine | 10 mg/kg | 20 mg/kg | 30 mg/kg |

Experiments are performed to test the maximum non-sedating dose combination (1.6/10 mg/kg) that was identified as conferring the best analgesic benefit using a diabetic rat model. Combinations are tested against:
1. multiples of the dose combinations.
2. against no drug (saline) and Flupirtine doses (10, 20, 30 mg/kg) without opioid.
3. against opioid doses (1.6 mg/kg, 3.2 mg/kg, 4.8 mg/kg) without co-administered Flupirtine

For experiments in which drugs are administered to rats by intraperitoneal injection, the following outcomes would be anticipated:
A FDC containing 10 mg/kg flupirtine and 1.6 mg/kg morphine is no more preferred, and preferably less preferred than a dose of 1.6 mg/kg morphine administered alone;
A FDC of 20 mg/kg flupirtine and 3.2 mg/kg morphine is no more preferred and preferably less preferred than a FDC containing 10 mg/kg flupirtine and 1.6 mg/kg morphine
A FDC of 30 mg/kg flupirtine and 4.8 mg/kg morphine is no more preferred and preferably less preferred than a FDC containing 10 mg/kg flupirtine and 1.6 mg/kg morphine

Test are also conducted using a FDC comprising flupirtine/opioid combinations with both lower and higher doses of morphine (or corresponding opioid) than those listed above.
e.g. 1.0 mg/kg morphine; and
   2.0 mg/kg morphine multiples.

These experiments are repeated using various concentrations of other exemplary opioids such as oxycodone, methadone, hydromorphone, and fentanyl. In addition, experiments are performed in which flupirtine has been substituted with retigabine. In each case, the maximum non-sedating doses of each candidate drug to be studied in the FDC are determined when administered alone and in combination

Comparative studies are also performed which demonstrate the abuse potential of combinations that are representative of alternative opioid/non-opioid fixed dose combination products (e.g. oxycodone/acetaminophen, hydrocodone/acetaminophen, oxycodone/ibuprofen) that are already available in the market and are known to be susceptible to abuse by patients and others who might seek to obtain this for non-therapeutic (abusive) uses are also tested. These experiments are performed using FDCs containing maximum non-sedating dose combinations of these drugs determined according to procedures detailed above and FDCs containing multiples of these dose combinations.

### Example 2

### Human Experiments to Demonstrate the Abuse Deterrent Properties of Analgesically Effective Fixed-Dose Combinations

Drug liking studies are conducted using human volunteers (preferably with a history of occasional opioid abuse and without significant medical or psychiatric disturbances) to determine whether a particular FDC is likely to be abused if it becomes publicly available once it receives marketing approval from pharmaceutical regulatory agencies such as the US FDA and the EMEA.

Subjects participate as outpatients on alternate days for the duration of the study protocol. A placebo is administered on the first day for the purposes of training and adaptation to experimental procedures: this session is excluded from statistical analysis.

The following doses of flupirtine alone or oxycodone alone or flupirtine in combination with morphine are tested in random order in a double-blind fashion over sequential experimental sessions.

Flupirtine alone 100 mg
Oxycodone alone: 2 mg
Oxycodone alone 5 mg
Oxycodone alone 7.5 mg
Flupirtine 100 mg combined with oxycodone 2 mg
Flupirtine 100 mg combined with oxycodone 5 mg
Flupirtine 100 mg combined with oxycodone 7.5 mg

Subject effect measures are presented on a computer with a key pad. After baseline measurements, subjects ingest a capsule containing one of the above doses of flupirtine, oxycodone or a combination of flupirtine with oxycodone. All capsules have the same visual appearance and physical feel. Subjective, physiological, and behavioural measures (described below) are collected hourly from subjects for 6 hours following ingestion of a capsule.

### Subject Rated Measures:

1. 100-point visual analogue scales marked at either end with “none’ and “extreme” to rate the degree of drug (i) effect (ii) good effects (iii) bad effects (iv) high (v) liking
2. 100-point visual analogue scales marked at either end with “none’ and “extreme” to rate the degree of the drugs following symptoms/side-effects (i) confusion (ii) light-headedness/dizziness (iii) sleepiness/tiredness (iv) difficulty concentrating (v) slurred speech (vi) numb/tingling feeling (vii) irritability/grumpiness (viii) blurred vision (ix) nausea (x) comfort (xi) relaxation (xii) drunkenness (xiii) nervousness (xiv) energy (xv) dry mouth
3. Addiction research centre inventory questionnaire (Martin et al 1971)
4. Profile of Mood States (McNair et al 1971)

It is anticipated that subjects will exhibit the following preferences:
Any particular dose of oxycodone administered alone is preferred to, or no less preferred to, the same dose of oxycodone administered in combination with 100 mg flupirtine.
Any particular dose of oxycodone administered in combination with 100 mg flupirtine is preferred to, or no less preferred to, twice that dose of oxycodone administered in combination with 200 mg flupirtine.
Any particular dose of oxycodone administered in combination with 100 mg flupirtine is preferred to three times that dose of oxycodone administered in combination with 300 mg flupirtine.

Similar experiments are conducted using morphine and other different opioids at doses equivalent to the oxycodone doses shown above (as calculated using MEDD factors shown in Table 1) and using retigabine in place of flupirtine (50, 100, 150 mg retigabine in place of 100 mg, 200 mg 300 mg flupirtine). Similar results are expected.

### BIBLIOGRAPHY

Bardo and Bevins Psychopharmacology (Berl) 153:31-43, 2000
Goodchild et al. Pain Medicine 9:928-938, 2008
Hoffmann et al. Brain Research Bulletin 23:373-387, 1989
Melzack et al. Ann NY Acad Sci, 933: 157-174, 2001
Pelloux et al. Physiology Biochemistry and Behaviour 84:43-60, 2006
Rajbhandari et al. Pain 83:627-629, 1999
Teng and Mekhail Pain Practice 3:8-12, 2003

1. A method for inducing an analgesic response to pain in a subject said method comprising administering to the subject an amount of an opioid and an agent selected from flupirtine and retigabine which is effective to reduce the sensation of pain whilst reducing the enticement for substance abuse.

2. The method of claim 1 wherein the opioid is morphine.

3. The method of claim 1 or 2 wherein the opioid is administered in an amount of from 0.1 mg to 200 mg of morphine or an equivalent dose of other opioid as determined using equivalent dose factors listed in Table 1.

4. The method of claim 3 wherein the flupirtine is administered in an amount of from 25 mg to 1000 mg.

5. The method of claim 3 wherein the retigabine is administered in an amount from 10 mg to 500 mg.

6. The method of claim 1 wherein the subject is human.

7. Use of an opioid and an agent selected from flupirtine and retigabine in the manufacture of an abuse deterrent, fixed dose composition.

8. Use of claim 7 wherein the opioid is morphine.

9. Use of claim 7 or 8 wherein the opioid is provided in an amount of from 0.1 mg to 200 mg of morphine or an equivalent dose of other opioid as determined using equivalent dose factors listed in Table 1.

10. Use of claim 9 wherein the flupirtine is provided in an amount of from 25 mg to 1000 mg.

11. Use of claim 9 wherein the retigabine is provided in an amount from 10 mg to 500 mg.

12. An abuse deterrent, fixed dose composition comprising an opioid in an amount of from 0.1 mg to 200 mg of morphine or other opioid equivalent as determined by Table 1, and an agent selected from 25 mg to 1000 mg flupirtine and 10 mg to 500 mg retigabine.

13. The composition of claim 12 further comprising one or more pharmaceutically acceptable excipients, carriers and/or diluents.

14. Use of the composition of claim 12 or 13 in the manufacture of a medicament for the treatment of pain.

15. A pain management protocol with a reduced enticement for substance abuse, said protocol comprising administering to a subject in need of pain management an amount of an opioid and an agent selected from flupirtine and retigabine which is effective to reduce the sensation of pain whilst reducing the enticement for substance abuse.

16. The protocol of claim 15 wherein the opioid is morphine.

17. The protocol of claim 15 or 16 wherein the opioid is administered in an amount of from 0.1 mg to 200 mg of morphine or an equivalent dose of other opioid as determined using equivalent dose factors listed in Table 1.

18. The protocol of claim 17 wherein the flupirtine is administered in an amount of from 25 mg to 1000 mg.

19. The protocol of claim 17 wherein the retigabine is administered in an amount from 10 mg to 500 mg.

20. The protocol of claim 15 wherein the subject is a human.
